(19) United States
(12) Patent Application Publication
(10) Pub. No.: US 2023/0420079 A1
(43) Pub. Date: Dec. 28, 2023

(54) DIGITAL ANIMAL FREE TESTING (DAFT) SYSTEM

(71) Applicants: Subhadra Dravida, Hyderabad (IN); Samhita B L N, Frisco, TX (US)

(72) Inventors: Subhadra Dravida, Hyderabad (IN); Samhita B L N, Frisco, TX (US)

(73) Assignee: TRANSCELL ONCOLOGICS PRIVATE LIMITED, Hyderabad, TG (IN)

(21) Appl. No.: 18/235,895

(22) Filed: Aug. 21, 2023

Related U.S. Application Data
(63) Continuation-in-part of application No. 17/722,528, filed on Apr. 18, 2022.

(30) Foreign Application Priority Data
Feb. 15, 2022 (IN) 202241008032

(51) Int. Cl. G16B 40/00; C12M 3/00

(52) U.S. Cl. CPC G16B 40/00; C12M 23/44

(57) ABSTRACT

The present invention discloses a workstation solution for test predicting human safety concerns and efficacy measurements in a test agent. The workstation solution comprises, a real-time platform or human MicroPhysiological Systems (hMPS) unit, and a digital platform. The digital platform with embedded artificial intelligence (AI) is configured to predict safety (pharmacology) risks from phenotypes, genotypes and proteotype data sets acquired from test agents treated hMPS platform in a modular assay system. The digital platform is trained with phenotypes, genotypes, proteotypes, biochemical data sets as benchmark patterns and signals configured as positive or negative controls to provide a bandwidth to AI for detecting anomalies in a real-time assaying and for measuring the analyzed insights in real-time assaying. The workstation solution is a Digital Animal Free Testing (DAFT), which is a foundational scheme while the modular assay system is one of a derived application.

### CROSS-REFERENCE TO RELATED APPLICATIONS

The present application is a continuation-in-part application of the non-provisional patent application titled “A NON-ANIMAL HUMAN RELEVANT WORKSTATION SYSTEM AND METHOD FOR TESTING NEUROVIRULENCE AND NEUROTOXICITY IN VACCINES”, application Ser. No. 17/722,528, filed in the United States Patent and Trademark Office on Apr. 18, 2022, which further claims priority to Indian Patent Application No. 202241008032 filed on Feb. 15, 2022, the entire contents of which are hereby incorporated by reference for all purposes.

### FIELD OF THE INVENTION

The present invention generally relates to the field of non-animal safety or efficacy testing strategies of any pharma or biopharmaceuticals developed for human consumption. Further, the present invention relates to a system and technology for cruelty-free testing that leverages human Microphysiological Systems (hMPS), Artificial Intelligence (AI), Machine Learning (ML) tools. More specifically, the present invention relates to Digital Animal Free Testing (DAFT), which is a foundational scheme while the modular assay system NeuroSAFE, as a Solution is one of the derived applications.

### BACKGROUND

The use of animals for various purposes, such as food, transportation, pets, sports, recreation, and companionship, has been an integral part of human history. However, the use of animals for scientific research, particularly in assessing safety risks and efficacy concerns of pharma and biopharmaceuticals, has raised ethical concerns due to the cruelty and suffering inflicted upon them. Animals ranging from small creatures like mice, rats, and rabbits to larger ones like primates and dogs have been extensively used in research for drug efficacy testing, safety-related toxicity screenings, and medical procedure investigations.

Despite the widespread use of animals in research, there are growing concerns about the reliability and predictive value of their outcomes for understanding human physiology and developing new treatments for human diseases. Three major conditions that undermine the confidence in animal experimentation for translating results to humans: (1) Effects of the laboratory environment and other variables: The experimental settings in laboratories may significantly influence study outcomes, leading to disparities between animal and human results, (2) Discordance between animal models of disease and human diseases: Animal models of human diseases, typically induced artificially, may not accurately represent the complexity and pathology of the corresponding human conditions, resulting in low translational reliability, and (3) Interspecies differences in physiology and genetics: Even if animal models closely resemble human diseases, fundamental differences in physiology, behavior, pharmacokinetics, and genetics can limit the reliability of animal studies in predicting human outcomes.

The high failure rate of translating results from animal experiments to successful clinical trials further emphasizes the need for alternatives to animal testing. Emerging non-animal methods, including the use of human cells and tissues, computer models, volunteer studies, and advancements in technology provide more relevant ways of studying human biology and disease.

Henceforth, there is need for an innovation to address the limitations and ethical concerns associated with animal experimentation by offering a new, human-based paradigm in medical research and drug development. Utilization of human Microphysiological Systems (hMPS), models and testing methods based on human biology, eliminates the guesswork required when extrapolating data from animals to humans. The use of these methods can lead to more accurate results, avoiding the potential abandonment of useful medical treatments based on misleading animal tests and the misdirection of resources. The strategy of the 3 Rs (reduction, refinement, and replacement) is also an approach to overcome the drawbacks of animal experiments. Reduction focuses on minimizing the number of animals used, refinement aims to improve animal welfare and reduce suffering, while replacement seeks to replace animal models with more relevant and ethical alternatives.

### FDA Modernization Act 2.0

The “FDA Modernization Act 2.0” recognizes the growing concerns and ethical considerations regarding the use of animals in testing for pharmaceuticals, medical devices, and other healthcare products. The act seeks to address these concerns by encouraging the development and adoption of alternative methods to animal testing while ensuring that the safety and efficacy of products are adequately assessed.

The act promotes the implementation of the Three Rs principle—Reduction, Refinement, and Replacement—in animal testing practices. The Three Rs principle emphasizes the need to: (1) Reduce the number of animals used in testing by employing alternative methods, such as in vitro cell cultures, computer modeling, and human-based testing techniques whenever possible, (2) Refine the testing protocols to minimize the pain, suffering, and distress experienced by animals during experiments. The act encourages researchers and companies to adopt more humane and sophisticated techniques that reduce the negative impact on animals, (3) Replace animal testing with alternative methods that are scientifically valid, reliable, and predictive of human responses. The act promotes the use of innovative technologies, such as organ-on-chip models, tissue engineering, and computer simulations, to replace animal experiments where appropriate.

The “1-DA Modernization Act 2.0” also encourages collaboration and information sharing between regulatory agencies, research institutions, and industry stakeholders to advance the development and validation of alternative testing methods. By promoting research into non-animal alternatives and providing resources for their validation, the act aims to create a robust scientific foundation for the acceptance of these methods in regulatory decision-making.

Additionally, the act emphasizes the importance of transparency in animal testing practices. It requires that researchers and companies using animals in testing provide clear justifications for their use, demonstrate efforts to minimize animal use and suffering, and disclose the results of their experiments to regulatory authorities.

Furthermore, the act acknowledges the role of international cooperation in promoting alternatives to animal testing. It encourages harmonization of regulatory standards and acceptance of validated alternative methods across different countries to avoid redundant testing and facilitate global market access for products.

“FDA Modernization Act 2.0” reflects a commitment to address the ethical concerns related to animal testing while maintaining the safety and efficacy standards for healthcare products. By promoting the Three Rs principle, encouraging research and validation of alternative methods, and fostering transparency and international collaboration, the act aims to promote the adoption of non-animal testing approaches and contribute to the advancement of ethical and scientifically sound practices in the healthcare industry.

Existing alternatives to animal testing, such as in vitro, ex vivo, and in silico protocols, represent innovative and ethical approaches that aim to replace or reduce the use of animals in scientific research and testing. These alternative methods provide valuable insights into human biology, disease mechanisms, and drug responses without the need for animal experimentation.

In vitro Testing:

In vitro testing involves conducting experiments using isolated cells, tissues, or organs outside of a living organism, usually in a laboratory setting. In vitro models offer several advantages, including the ability to control experimental conditions, reduce animal usage, and provide rapid results. Some common in vitro methods include: (a) Cell Cultures: Cultured human cells, such as human cell lines or primary cells, can be used to assess cellular responses to various compounds, drugs, or toxins, (b) Organoids: Organoids are three-dimensional cell culture systems that mimic the structure and function of specific organs, offering a more physiologically relevant model for drug testing and disease research, (c) Tissue Chips: Organ-on-a-chip or tissue chip technologies use microfluidic devices to create small-scale models of human organs, enabling the study of organ-level interactions and responses to drugs or diseases.

Ex vivo Testing:

Ex vivo testing involves conducting experiments using intact organs or tissues that have been removed from a living organism. These tissues are maintained in a controlled environment that allows researchers to study their responses without the need for live animals. Some examples of ex vivo methods include: (a) Human Tissue Studies: Human tissues obtained from biopsies, post-mortem samples, or surgical procedures can be used to study human-specific responses to drugs and diseases (b) Isolated Organ Studies: Whole organs, such as liver or heart, can be isolated and maintained in a laboratory setting for drug metabolism and toxicity testing, (c) In silico Testing: In silico testing involves using computer-based models and simulations to predict biological responses, drug interactions, and toxicity. These methods leverage computational algorithms and databases to model human physiology and predict outcomes without actual experimentation on animals. Some examples of in silico methods include: (1) Computational Models: Mathematical models of biological processes, pharmacokinetics, and pharmacodynamics can be developed to simulate drug interactions and predict their effects on the human body. (2) Molecular Docking: Molecular docking simulations can predict how a drug molecule will bind to its target protein, providing valuable insights into drug-receptor interactions. (3) High-Throughput Screening: Virtual screening techniques can analyze vast databases of chemical compounds to identify potential drug candidates or assess their toxicity.

Using transformed cell lines in in vitro testing protocols has certain disadvantages. While transformed cell lines offer several benefits, such as ease of maintenance, reproducibility, and availability, they also come with certain limitations that can affect the validity and relevance of experimental results. Some known disadvantages of using transformed cell lines in in vitro testing protocols include:
(1) Loss of Normal Cellular Functions: Transformed cell lines are typically derived from cancerous or immortalized cells, which have undergone genetic alterations to promote uncontrolled cell growth. As a result, these cells may lose some of the normal cellular functions and responses that are present in non-transformed cells. This alteration can lead to differences in drug metabolism, signaling pathways, and other cellular processes, making the results less reflective of physiological conditions in the human body.
(2) Lack of Tissue-Specificity: Transformed cell lines are often derived from a specific cell type, such as breast cancer cells or lung cancer cells. However, they may not fully represent the complexity and heterogeneity of the actual tissues or organs they are supposed to mimic. This lack of tissue-specificity can limit the relevance of experimental data when studying drug responses or disease mechanisms.
(3) Limited Representation of Cell-Cell Interactions: In vivo, cells within tissues and organs interact with each other through complex signaling networks. Transformed cell lines in in vitro settings may not fully replicate these intercellular interactions, leading to an oversimplified representation of cellular responses and outcomes.
(4) Genetic Drift and Phenotypic Variability: Transformed cell lines can undergo genetic drift over time, leading to changes in their genetic makeup and phenotypic characteristics. This drift can result in variations between different batches of the same cell line, affecting the reproducibility of experiments.
(5) Susceptibility to Contamination: Transformed cell lines are more prone to contamination issues, such as mycoplasma contamination, which can alter experimental results and introduce confounding factors.
(6) Lack of 3D Architecture: Many in vitro tests using transformed cell lines are conducted on two-dimensional (2D) culture systems. However, cells in vivo often exist in three-dimensional (3D) architectures within tissues. This difference in cell organization can impact cellular behavior, drug response, and cellular signaling.
(7) Limited Physiological Relevance: Transformed cell lines may not accurately reflect the physiological conditions found in healthy human tissues, limiting their utility for certain types of studies, such as toxicity assessments, drug metabolism, and tissue-specific responses.
(8) Potentially Biased Drug Response: Transformed cell lines may have unique characteristics and molecular profiles that bias their response to drugs differently from normal cells or tissues. This bias can lead to inaccurate predictions of drug efficacy and safety.

However, using primary and progenitor cell/tissue-based in vitro or ex vivo hMPS and methods offer several advantages over transformed cell lines or traditional in vivo animal models. These methods involve using cells or tissues directly sourced from human or animal donors without genetic manipulation, providing a more physiologically relevant and accurate representation of the actual biological systems. Here are some of the advantages of using primary and progenitor cell/tissue-based in vitro or ex vivo methods: (1) Increased Physiological Relevance: Primary and progenitor cells/tissues closely resemble the cells found in the human body or specific tissues of interest. This similarity allows researchers to study cellular responses, signaling pathways, and drug interactions under conditions that more accurately mimic the in vivo environment, enhancing the translatability of research findings to humans, (2) Retention of Native Cellular Functions: Unlike transformed cell lines, primary and progenitor cells often retain their native cellular functions, including cell-cell interactions and responses to physiological cues. This enables researchers to investigate complex cellular processes and signaling networks that may be absent or altered in transformed cell lines, (3) Tissue-Specificity: Primary and progenitor cell/tissue-based models provide researchers with the opportunity to study specific tissues or organs in isolation, allowing for a more targeted examination of disease mechanisms or drug responses in the context of a particular tissue microenvironment, (4) Personalized Medicine Applications: Primary cells derived from individual patients, such as patient-derived tumor cells or stem cells, can be used for personalized medicine applications. These cells can be used to screen potential treatments and determine personalized therapeutic approaches based on a patient's unique genetic makeup and disease profile. (5) Cellular Diversity: Primary and progenitor cell/tissue-based models inherently possess cellular diversity, which is essential for capturing the heterogeneity observed in human tissues. This diversity allows researchers to study intercellular interactions and cellular responses that may be overlooked in transformed cell lines. (6) Drug Metabolism and Toxicity Assessment: Primary and progenitor cell/tissue-based models are valuable tools for assessing drug metabolism and toxicity more accurately. These models can provide insights into drug interactions with specific tissues and organs, aiding in the prediction of drug safety and efficacy in humans. (7) Reduced Contamination Risk: Primary and progenitor cells are obtained directly from donors and are typically less prone to contamination issues compared to transformed cell lines, which are often passaged and maintained in culture for extended periods. (8) Ethical Considerations: The use of primary and progenitor cells/tissues reduces the reliance on animal models, aligning with ethical considerations in research and contributing to more humane experimental practices. (9) Preclinical Drug Testing: Primary and progenitor cell/tissue-based models can be used for preclinical drug testing, allowing researchers to assess drug candidates in a more relevant biological context before proceeding to animal studies or clinical trials. (10) Advancements in Organ-on-a-Chip Technology: Organ-on-a-chip and tissue-on-a-chip technologies leverage primary and progenitor cells to create microphysiological systems that mimic specific organs or tissues, enabling the study of organ-level responses to drugs, diseases, and environmental cues.

Therefore, according to the present invention now, DAFT combines in vitro or ex vivo platform composed of primary, progenitor human sourced cell/tissue configurations, hMPS with AI/ML powered in silico protocols to assess either safety or efficacy assessments. DAFT is a New Approach Methodology (NAM) based technology that leverages relevant hMPS model (proprietary configurations) and AI/ML powered digital platform (proprietary software). DAFT's hMPS are human induced pluripotent stem cell (hiPSC) configured platform or hiPSC derived lineage specific cellular moieties or primary cells or primary progenitor cellular moieties mimicking human physiology. The relevant hMPS model composition and the assaying protocol would dictate the use case of DAFT: For e.g., if hMPS is hiPSC derived neurons, it is NeuroSAFE as the modular assay for test predicting human neurovirulence or neurotoxicity. If hMPS is hiPSC derived cardiomyocytes, it is CardioSIGHT as the modular assay for test predicting cardiotoxicity. If hMPS is hiPSC derived hepatocytes milieu, it is HepatoSIGHT for test predicting hepatotoxicity and Drug Induced Liver Injury (DILI). If hMPS is human hematopoietic ex vivo system, it is ImmunoGEN as the modular assay for measuring antigenicity and immunogenicity like readouts.

Further, in general, a vaccine is a biological preparation that provides immunity to a particular infectious disease. Vaccines greatly reduce the risk of infection by working with the body's natural defenses to safely develop immunity to disease. The vaccine contains an agent that resembles a disease-causing microorganism and is often made from weakened or killed forms of the microbe, its toxins, or one of its surface proteins. The agent stimulates the body's immune system to recognize the agent as a threat, destroy it, and to further recognize and destroy any of the microorganisms associated with that agent that it may encounter in the future.

A report states that the vaccines have been the single most fruitful investment in public health after sanitation and clean water and have contributed to drastically reducing the incidence and mortality ensuing from pathogenic and communicable diseases. It is no wonder that some of the world's largest philanthropies have invested heavily in these instruments, alongside state public health agencies. New pathogens will emerge in every era, and vaccines are likely to be the best bet against them. Nowhere has this been so starkly demonstrated as in the COVID-19 pandemic.

Vaccines are of various types, and their variety has proliferated in tandem with advances in the chemical, physical, biological sciences, and computational sciences. These comprise, in addition to the conventional types such as attenuated and live inactivated viral vaccines, viral-vectored vaccines, and subunit vaccines, newer forms like the viral-like particle vaccines, nanoparticle vaccines, recombinant proteins, polysaccharide-based immunogens, hybrid molecules (part engineered and part native), DNA/RNA vaccines, and the rationally-designed vaccines. Vaccine-induced prophylaxis is now available for non-communicable diseases (NCDs) such as diabetes, cancer, rheumatoid arthritis, and cardiovascular disease.

The newer vaccines are designed to be immunogenic and to simultaneously reduce the likelihood of adverse events. Their presence notwithstanding, inactivated and the live attenuated virus (LAV) vaccines continue to be part of the prophylactic arsenal of state public health services, as they are known to be effective, and industrial systems are already set up for their manufacture. The LAV comprises “live” viruses that can reproduce normally but have lost the ability, through mutations, to cause disease. Because they can multiply and amplify their titre in the host circulation over a period, the immune challenge is large enough, and long enough, for a single dose to suffice. Their ability to multiply in the host confers on them a peculiar property, and this is described below.

Neurotropic viruses and Neurovirulence:

Viruses are fastidious beings; in that they target and affect specific tissues in specific organisms. This affinity to a specific tissue, in a specific host, is termed tropism. Neurotropic viruses therefore are those which selectively infect nervous tissue; they have evolved genetically to perform the various preliminary steps to successful neuroinvasion, and subsequent multiplication within neural or neuronal cells, to cause neurovirulence. Neurotropic viruses may enter the central nervous system through the blood-brain barrier, or “centripetally” through the peripheral nervous system. They may enter neuronal cells and establish latency, or cause apoptotic neural damage through the lytic pathway as shown in FIG. 1. Neurotropic viruses must counter the host's innate immune response to infection, inhibit autophagy of cells they infect, and reverse the host-directed shutdown of the protein synthetic machinery, and there are variations on this theme, depending on the virus. Because neurovirulence targets the central nervous system (CNS) it often manifests clinically as encephalitis.

The list of neurotropic viruses is veritable who's who of the most pernicious pathogens known to man. Cytomegalovirus, influenza viruses (including the human coronaviruses), and the viruses causing polio, Yellow Fever, Japanese Encephalitis, mumps, measles, rabies, herpes, and HIV. A large proportion of emerging viruses are neurotropic and can cause serious neurological disease, SARS-COV-2 causing Covid-19 being the most recent addition to this morbidly important group.

FIG. 1 shows the tendency or capacity of the vaccine to cause or trigger disease of the nervous system 100. The neurons 102 in the CNS are affected by SARS-COV and SARS-COV-2 virus 104 causing viral invasion in the CNS. The SARS-COV and SARS-COV-2 virus 104 affect the cell membrane 106 causing neural infection, where the cell membrane comprises an angiotensin-converting enzyme 2 (ACE2) 108 and a cell surface protein transmembrane serine protease 2 (TMPRSS2) 110. The virus entered the nervous systems cause neural damages 112 i.e., Immune-mediated CNS damage in the CNS.

The Guillain Barre syndrome, caused by Johnson and Johnson's single-shot vaccine is the most recent instance, of several, vaccine-derived neurovirulence. The neurotropic virus vaccines introduced into the market since 2001 number more than 60, and underscore the importance of this safety check. For this reason, international and regional laws require that vaccine lots be assiduously checked for neurovirulence before releasing for sale and that pharmacovigilance be enforced. A reliable testing mechanism for reacquisition of neurovirulence in the vaccine batch production will go far in mitigating this problem.

Mutations responsible for live-virus attenuation have not been fully characterized, except for the oral polio vaccine (OPV). Viruses have much higher rates of mutations than do bacteria or higher life forms, and RNA viruses more than DNA viruses. Attenuation requires different numbers of passages for different viruses, and this is peculiar to the virus; some require a few tens, others several hundred. Further, different numbers of attenuating mutations are present in different live attenuated viruses, and those with a larger number are less likely to revert. The quality that confers attenuation can also cause spontaneous reversion to the virulent wildtype form. Evolution cuts both ways. A reversion may be through back-mutations, compensatory mutations in different regions of the genome, and recombination with other viruses. About 1 in 750,000 children who are vaccinated with the OPV are afflicted with vaccine-derived polio. Studies with the oral polio vaccine have also shown that reversion rates are appreciable and that they depend on the immunization schedule and the route of administration of the vaccine. The propensity of the virus to revert to a virulent form is such that the live-attenuated form of the poliovirus vaccine has been completely replaced by the inactivated form.

### The Monkey Neurovirulence Test Methodology:

If viruses intended for vaccine manufacture are naturally neurotropic, or bear components that are neurotropic, or have been passaged through neuronal cells, regulations require that they be assessed for not just general virulence, but also neurovirulence. Neuroattenuation must be assessed and demonstrated, and this generally takes the form of the Monkey Neurovirulence Test (MNVT) as shown in FIG. 2.

To reiterate, the vaccine Master Lots are required by law to be tested by the MNVT; the USFDA requires that 5 of these Master Lots be tested for neuroattenuation, or neurovirulence. Subsequent lot testing is generally not followed once the master lots are checked. The frequency of testing post the Master Lot checks depends on the regulatory agency: the World Health Organisation (WHO), the Japanese PMDA, the American Food and Drug Administration (USFDA), or the EMA.

The MNVT takes the form of a regular experiment. Each test requires approximately 30 monkeys; three groups of monkeys are used, one as a negative control that does not receive any virus, one as a positive control that receives a virulent form, and one test group that receives the candidate vaccine. Candidate virus is inoculated into the brain or spinal cord of monkeys of the Macaca or Cercopithecus genera and animals observed for symptoms of neural damage over a 17 to 22-day period. Monkeys were traditionally monitored for clinical signs of encephalitis, and this practice continues. In addition, the monkeys are also euthanized and examined by histopathology for viral lesions in the brain and spinal cord tissue. Three regions of the brain are of interest: the target regions, which are inflamed upon infection by both neurovirulent and non-neurovirulent viruses, the eponymous discriminator regions, which are preferentially infected by the neurovirulent viruses, and control regions that are not affected. The degree of neurovirulence is inferred from glial cell activation and infiltration of the CNS by primary immune cells, and these cellular events are semi quantitatively scored.

The MNVT as it is called, has been the neurovirulence test of choice for poliomyelitis, measles, mumps, rubella, varicella, influenza, yellow-fever viruses, and more recently for the COVID vaccines, the rationale for the model being the phylogenetic proximity of human and nonhuman primates. The rationale is questionable; there is a certain dissonance between the MNVT's persistence in vaccine safety testing and the fundamental flaw in it, viz that genetic relatedness does not always translate into the relevance and predictive value (although this is a general caveat with any model system). A lack of a resemblance between the simian cell surface structures, which the virus uses to gain entry, can preclude the MNVT's relevance to the virus and render it useless. Instances of the MNVT's inability to detect neurovirulence exist. For mumps vaccines, the MNVT only showed non-significant trends towards differences between wild-type and attenuated mumps viruses and failed to detect residual neurovirulence in the Urabe Am9 strain of mumps vaccine. This strain was developed in 1967 based on a Japanese isolate of mumps virus, passaged through chicken and quail cells. The vaccine was widely distributed in Canada, Japan, and Europe until cases of vaccine-associated aseptic meningitis were detected in Canada. More cases were found in Japan and the UK, with an estimated 38-330 cases of aseptic meningitis per 100,000 vaccine recipients. Following these findings, the Urabe Am9 vaccine saw reduced use, and in Japan, mumps was removed as a routine vaccine altogether. Following this policy change, Japan has seen a surge in mumps cases, with up to 1.5 million infections annually.

### Alternatives to the MNVT:

The MNVT was conceived at a time when comparable model systems did not exist, but the scientific and technological context has changed now, and alternatives should be given serious consideration. Efforts have been ongoing for years now to expand the repertoire of alternatives, but R&D alternatives to the MNVT have been slow coming. The primary reason is technical: it is difficult to develop a model that resembles the real “thing” (in this case, the human) to the degree that it is predictive of the actual response. Monkeys and (transgenic) mice are good models to the degree that their cellular and molecular anatomy resembles the humans', i.e., if the cell-surface structures facilitating viral entry are the same in the two. The technical problems notwithstanding, non-human primates other than monkeys have been used, successfully, for some vaccines, including the Ebola virus, Zika virus, alphaviruses, and influenza virus (Fulton and Bailey). Mice are genetically better characterized than non-human primates and have a greater number of pharmacological endpoints or biomarkers. Neonatal mice, for instance, appear to be supremely sensitive to some human viral pathogens and have been successfully used as models. Although mice appear to be a model to the NVT for the screening of live attenuated influenza vaccines, and transgenic mice constitute the single model, as an alternative for residual neurovirulence testing in poliovirus vaccines. It is, however, cruel, equally cumbersome and the readouts have to be extrapolated to healthy human physiology.

Though various systems and methods exist for testing neurovirulence in assuring vaccine safety and immunogenicity like efficacy assessments, they are animal based, time consuming, burdensome associated with erratic and faulty results for batch release testing requirements. Also, the test results are extrapolated to humans and have occupational hazards, and are undertaken only for regulatory submission. In addition, FIG. 3 shows a process flow 300 for ImmunoGEN's assaying protocol to measure biopharmaceutical's efficacy on hMPS model and to be fed into digital platform's prediction model for immunogenicity report.

More specifically, in the present invention, the technology of Digital Animal Free Testing (DAFT), is the foundational scheme used as background wireframe. Further, there is a need for DAFT like strategy to be adopted by the global pharma and biopharmaceutical players to align with FDA Modernization Act 2.0 in testing safety or efficacy like concerns following non-animal testing methods, that covers at least 40 different applications (Safety & Efficacy, Potency testing like CardioSIGHT, HepatoSIGHT, ImmunoGEN, etc., including NeuroSAFE). DAFT will be useful to New drug discovery programs (discovery, preclinical stage), Pharmaceuticals (discovery, preclinical, clinical trials, manufacturing stages), Biopharmaceuticals (discovery, preclinical, clinical trials, manufacturing stages), Biosimilars (discovery, preclinical, clinical trials, manufacturing stages), Biologics (discovery, preclinical, clinical trials, manufacturing stages), Antivenoms (routine testing in production stage), Cosmetics & cell-gene therapy products (discovery, preclinical, clinical trials, manufacturing stages).

### SUMMARY OF THE INVENTION

The present invention generally relates to a system and method for cruelty-free testing of safety risks or efficacy concerns surrounding pharma and biopharmaceuticals developed for human consumption.

The present invention is a Digital Animal Free Testing (DAFT), which is a foundational scheme while NeuroSAFE Solution (as defined/disclosed in U.S. patent parent application Ser. No. 17/722,528) is one of the derived applications. Further, DAFT covers at least 40 different applications (Safety & Efficacy, Potency testing like CardioSIGHT, HepatoSIGHT, ImmunoGEN, etc., including NeuroSAFE). DAFT is useful to New drug discovery programs (discovery, preclinical stage), Pharmaceuticals (discovery, preclinical, clinical trials, manufacturing stages), Biopharmaceuticals (discovery, preclinical, clinical trials, manufacturing stages), Biosimilars (discovery, preclinical, clinical trials, manufacturing stages), Biologics (discovery, preclinical, clinical trials, manufacturing stages), Antivenoms (routine testing in production stage), Cosmetics (discovery, preclinical, clinical trials, manufacturing stages).

In one embodiment, the present invention discloses a workstation solution for test predicting human safety concerns in a test agent. The workstation solution for test predicting human safety concerns in a test agent, comprises: a real-time platform or hMPS unit, and a digital platform with embedded artificial intelligence (AI) and machine learning (ML) tools, augmented with robotic process automation framework. The real-time platform or human MicroPhysiological Systems (hMPS) unit is configured to incubate the test agent aliquots collected from the preparation. The digital platform with embedded artificial intelligence (AI) and machine learning (ML) tools, augmented with robotic process automation framework. Further, the digital platform with embedded artificial intelligence (AI) and machine learning (ML) tools, augmented with robotic process automation framework, is configured to predict safety (pharmacology) risks from one or more phenotypes, genotypes and proteotype data sets acquired from one or more test agents treated human MicroPhysiological Systems (hMPS) platform in a modular assay system. Further, the digital platform is trained with one or more phenotypes, genotypes, proteotypes, biochemical data sets as one or more benchmark patterns and signals configured as one or more positive or negative controls to provide a bandwidth to the digital platform with embedded artificial intelligence (AI) and machine learning (ML) tools for detecting the anomalies in a real-time assaying. Moreover, the workstation solution is a Digital Animal Free Testing (DAFT), which is a foundational scheme while the modular assay system is one of a derived application.

In another embodiment, the present invention discloses a workstation solution for test predicting efficacy measurements in a test agent. The workstation solution for test predicting efficacy measurements in a test agent, comprises: a real-time platform or hMPS unit, and a digital platform with embedded artificial intelligence (AI) and machine learning (ML) tools, augmented with robotic process automation framework. The real-time platform or human MicroPhysiological Systems (hMPS) unit is configured to incubate the test agent aliquots collected from the preparation. The digital platform with embedded artificial intelligence (AI) and machine learning (ML) tools, augmented with robotic process automation framework. Further, the digital platform with embedded artificial intelligence (AI) and machine learning (ML) tools, augmented with robotic process automation framework, is configured to predict safety (pharmacology) risks from one or more phenotypes, genotypes and proteotype data sets acquired from one or more test agents treated human MicroPhysiological Systems (hMPS) platform in a modular assay system. Further, the digital platform is trained with one or more phenotypes, genotypes, proteotypes, biochemical data sets as one or more benchmark patterns and signals configured as one or more positive or negative controls to provide a bandwidth to the digital platform with embedded artificial intelligence (AI) and machine learning (ML) tools for measuring the analyzed insights in real-time assaying. Moreover, the workstation solution is a Digital Animal Free Testing (DAFT), which is a foundational scheme while the modular assay system is one of a derived application.

Digital Animal Free Testing (DAFT) leverages human Microphysiological System (hMPS) and AI (Artificial Intelligence)/ML (Machine Learning) tools, which are industrialized as assay systems with robotic process automation in reporting the readouts. Further, DAFT's digital wireframe has scope to add unlimited modules as assays.

The advantages or benefits of adopting DAFT are: (1) With one adoption in the workflow, can support the End User's discovery, preclinical and manufacturing stage bioassay or testing requirements, (2) Assays are embedded as modules to pick and choose, (3) Module wise input and output data is stored in access restricted cloud computing block chain framework, (4) Assays are robust in predicting the concerns, risks and requires no extrapolation to human species, (5) Assays can be performed in-house on relevant hMPS units supplied as Master banks for End User's exclusive usage, and (6) The prediction model will be trained with Reference drug/raw material/intermediate/adjuvant related phenomics data as control panel.

The system is a computer-implemented process executed in a network environment for testing the test aliquot. The system runs in the computer-implemented environment configured to provide a workstation solution that test predicts human safety risks or efficiency measurements. In one embodiment, the system utilizes a human biological discard sourced configured in vitro primary and progenitor cellular moieties and a process automation enabled digital platform to build AI-powered workstation solution to test and predict Immunogenicity. In one embodiment, the system is a cruelty-free DAFT solution that monitors the Phenotype/Genotype/Proteotype inconsistency of healthy human stem cell-based hematopoietic system treated with monoclonal antibody. In one embodiment, the system performs a non-animal, rapid immunogenicity test as a process-related assessment by the biopharmaceutical industry. In one embodiment, the system involves a non-clinical efficacy assessment as a workflow process in evaluating immunogenicity during the research and development (R&D), clinical trials, and production for immunization or therapeutic applications.

In one embodiment, the system comprises a real-time hMPS or TRANS-HSC platform and the prediction model. The TRANS-HSC is a phenotypically responsive, genotypically reactive, functionally readable configured, characterized human hematopoietic in vitro system, amenable to batch-wise large-scale production. In one embodiment, the TRANS-HSC is a human biological discard-sourced configured in vitro hematopoietic progenitor cells-based microphysiological platform to build a digital immunogenicity testing podium. In one embodiment, the TRANS-HSC is configured to incubate the test aliquots collected from the produced batches in the workflow.

In one embodiment, the digital platform or ImmunoGEN Solution is embedded with one or more artificial intelligence (AI) and machine learning (ML) modules, augmented with a robotic process automation framework. The AI modules are configured to measure antibodies produced from the hMPS culture supernatants.

In one embodiment, the network environment comprises one or more user devices. Each user device is associated with a user. In one embodiment, the user device is installed with a digital platform (i.e., ImmunoGEN).

In one embodiment, the immunogenicity evaluating system comprises a computing device and one or more databases in communication with the computing device. In one embodiment, the computing device is a server. In one embodiment, the computing device could be a cloud server. In one embodiment, the database is in communication with the computing device via the network. In one embodiment, the database is accessible by the computing device. In one embodiment, the databases are configured to store a plurality of reference data.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1 shows the tendency or capacity of the vaccine to cause or trigger disease of the nervous system.

FIG. 2 shows a monkey neurovirulence test methodology (MNVT).

FIG. 3 shows a process flow for ImmunoGEN's assaying protocol with the readouts fed into digital platform's prediction model to report immunogenicity of the test agent.

FIG. 4 shows an exemplary calibration and validation data sets of Digital Animal Replacement Technology (DAFT), which are processed by leveraging human Microphysiological System (hMPS) and AI/ML tools.

FIG. 5-6 shows an exemplary TRANS-HSC prepared from biological discards.

FIG. 7 shows an exemplary digital platform framework of DAFT.

FIG. 8 shows an exemplary DAFT's digital wireframe and its capability to add unlimited modules.

FIG. 9 shows a computer-implemented system executed in a network environment.

FIG. 10 shows example modular assays under DAFT with advantages to the organizations that adopt DAFT.

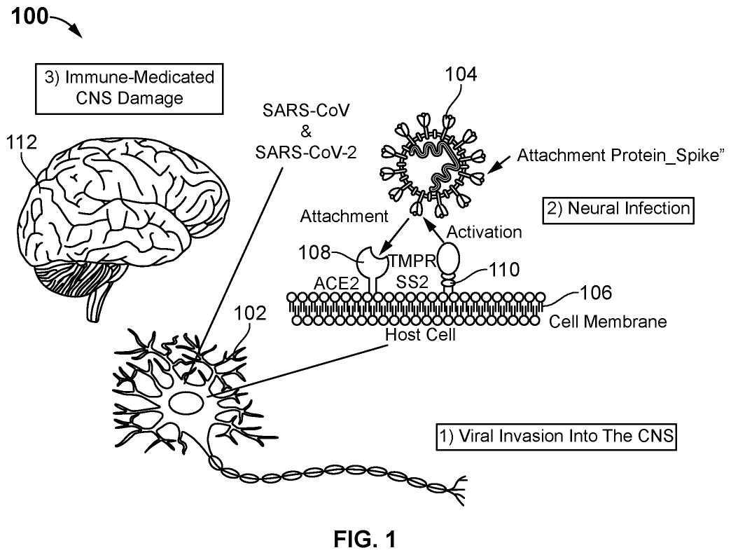

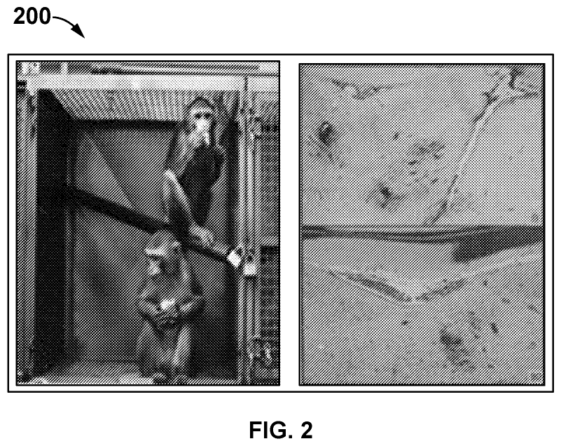

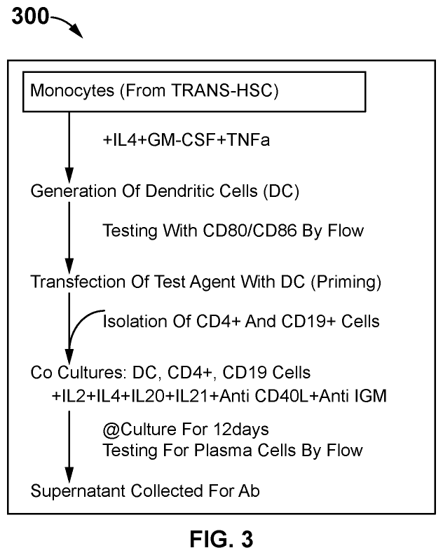

### DETAILED DESCRIPTION OF EMBODIMENTS

The present invention is best understood by reference to the detailed figures and description set forth herein. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

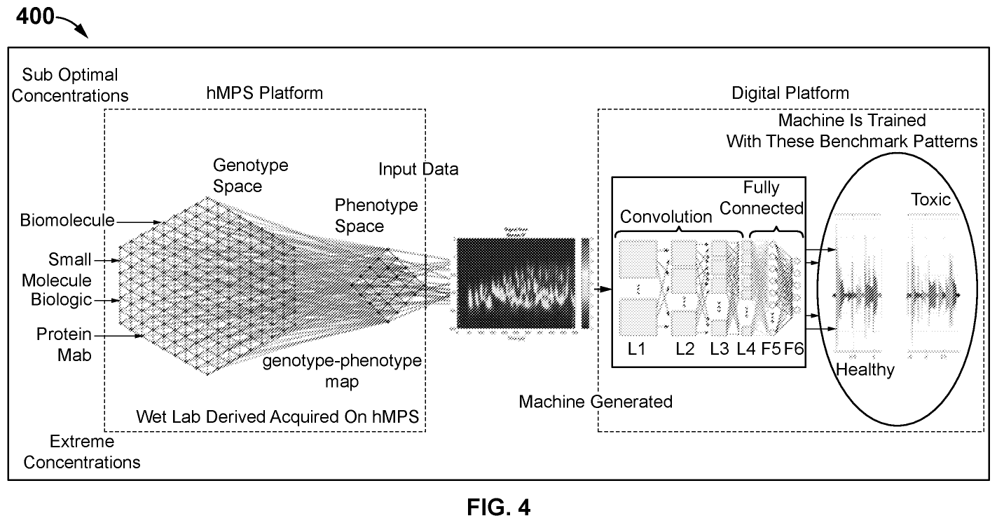

Referring to FIG. 4, an exemplary calibration and validation data sets of Digital Animal Replacement Technology (DAFT) 400, which are processed by leveraging human Microphysiological System (hMPS) and AI/ML tools, is disclosed. Digital Animal Free Testing (DAFT), which is a foundational scheme while the digital platform or NeuroSAFE Solution (as defined/disclosed in US patent parent application Ser. No. 17/722,528) is one of the derived applications. Digital Animal Free Testing (DAFT) leverages human Microphysiological System (hMPS) and AI (Artificial Intelligence)/ML (Machine Learning) tools, which are industrialized as assay systems with robotic process automation in reporting the readouts.

In one embodiment, the present invention discloses a workstation solution for test predicting human safety concerns in a test agent. The workstation solution for test predicting human safety concerns in a test agent, comprises: a real-time platform or hMPS unit, and a digital platform with embedded artificial intelligence (AI) and machine learning (ML) tools, augmented with robotic process automation framework. The real-time platform or human MicroPhysiological Systems (hMPS) unit is configured to incubate the test agent aliquots collected from the preparation. The digital platform with embedded artificial intelligence (AI) and machine learning (ML) tools, augmented with robotic process automation framework. Further, the digital platform with embedded artificial intelligence (AI) and machine learning (ML) tools, augmented with robotic process automation framework, is configured to predict safety (pharmacology) risks from one or more phenotypes, genotypes and proteotype data sets acquired from one or more test agents treated human MicroPhysiological Systems (hMPS) platform in a modular assay system. Further, the digital platform is trained with one or more phenotypes, genotypes, proteotypes, biochemical data sets as one or more benchmark patterns and signals configured as one or more positive or negative controls to provide a bandwidth to the digital platform with embedded artificial intelligence (AI) and machine learning (ML) tools for detecting the anomalies in a real-time assaying. Moreover, the workstation solution is a Digital Animal Free Testing (DAFT), which is a foundational scheme while the modular assay system is one of a derived application.

In another embodiment, the present invention discloses a workstation solution for test predicting efficacy measurements in a test agent. The workstation solution for test predicting efficacy measurements in a test agent, comprises: a real-time platform or hMPS unit, and a digital platform with embedded artificial intelligence (AI) and machine learning (ML) tools, augmented with robotic process automation framework. The real-time platform or human MicroPhysiological Systems (hMPS) unit is configured to incubate the test agent aliquots collected from the preparation. The digital platform with embedded artificial intelligence (AI) and machine learning (ML) tools, augmented with robotic process automation framework. Further, the digital platform with embedded artificial intelligence (AI) and machine learning (ML) tools, augmented with robotic process automation framework, is configured to predict safety (pharmacology) risks from one or more phenotypes, genotypes and proteotype data sets acquired from one or more test agents treated human MicroPhysiological Systems (hMPS) platform in a modular assay system. Further, the digital platform is trained with one or more phenotypes, genotypes, proteotypes, biochemical data sets as one or more benchmark patterns and signals configured as one or more positive or negative controls to provide a bandwidth to the digital platform with embedded artificial intelligence (AI) and machine learning (ML) tools for measuring the analyzed insights in real-time assaying. Moreover, the workstation solution is a Digital Animal Free Testing (DAFT), which is a foundational scheme while the modular assay system is one of a derived application.

Further, DAFT covers at least 40 different applications (Safety & Efficacy, Potency testing like CardioSIGHT (for cardiotoxicity), HepatoSIGHT (for hepatotoxicity), ImmunoGEN (for immunogenicity), etc., including NeuroSAFE). DAFT is useful to New drug discovery programs (discovery, preclinical stage), Pharmaceuticals (discovery, preclinical, clinical trials, manufacturing stages), Biopharmaceuticals (discovery, preclinical, clinical trials, manufacturing stages), Biosimilars (discovery, preclinical, clinical trials, manufacturing stages), Biologics (discovery, preclinical, clinical trials, manufacturing stages), Antivenoms (routine testing in production stage), Cosmetics (discovery, preclinical, clinical trials, manufacturing stages).

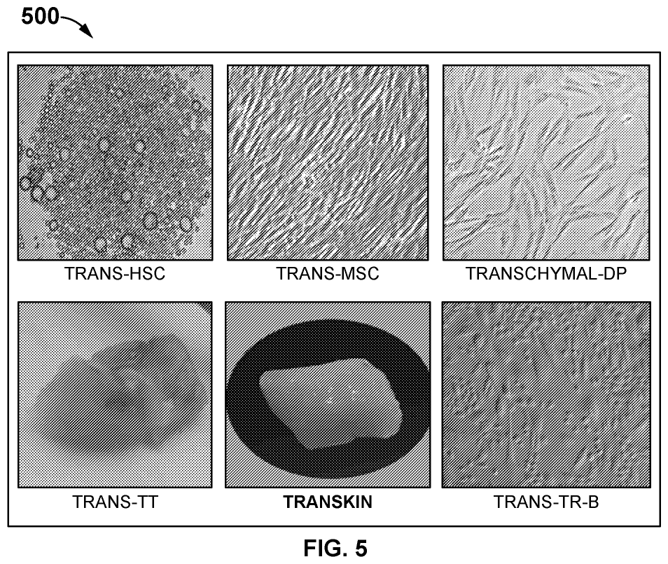

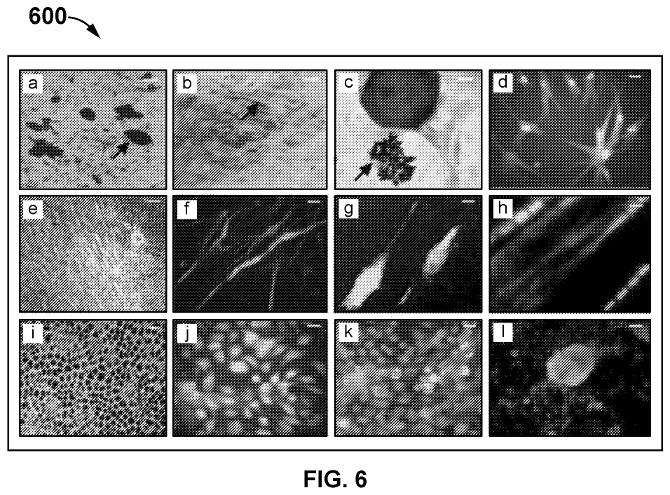

Referring to FIG. 5-6, an exemplary TRANS-HSC prepared from biological discards [500 & 600], is disclosed. (a) TRANS-MSC: Human induced Pluripotent Stem Cell (HiPSC) System, (b) TRANS-HSC: Human Hematopoietic in vitro System, (c) TRANSCHYMAL-DP: Human multipotent adult Mesenchymal Stem Cell System from Dental Pulp, (d) TRANSCHYMAL-AD: Human multipotent adult Mesenchymal Stem Cell System from Adipose, (e) TRANSCHYMAL-UC: Human multipotent adult Mesenchymal Stem Cell System from Umbilical Cord, (f) TRANS KIN: Human reengineered Skin construct, and TRANS-TT: Patient Tumor Tissue platform.

Some of the significant features of the human MicroPhysiological Systems (hMPS) in DAFT are as follows: (1) Complementary to existing transformed cell lines, (2) Specially configured in vitro platforms composed of primary, progenitors cells/tissues configured, (3) Platform composition dictating the use case—phenotypically responsive, genotypically reactive, and functionally approachable, (4) Relevance to human milieu, (5) Meaningful modeling, (6) Easy handling/predicting, and (7) No extrapolation of platform yielded readouts to human species.

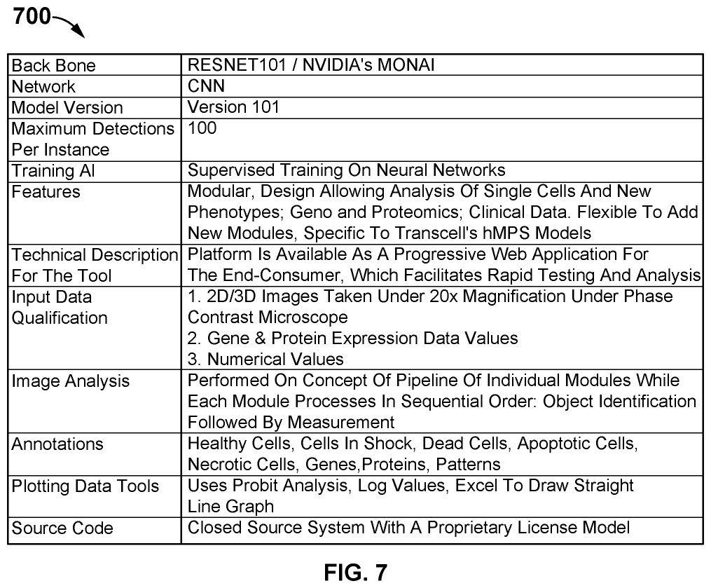

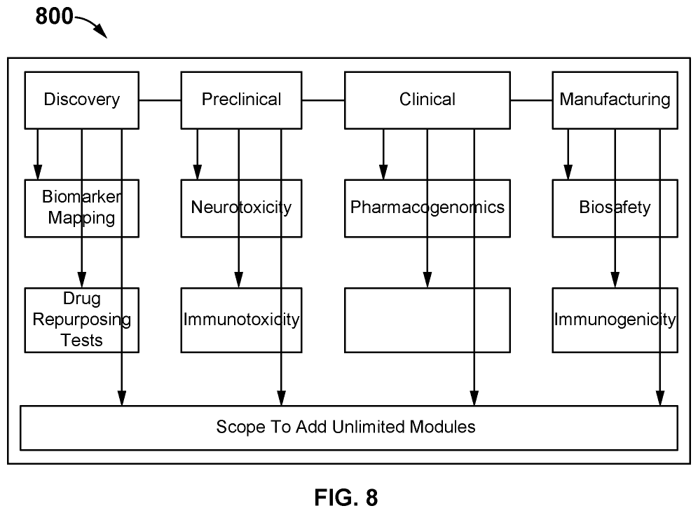

Referring to FIG. 7, an exemplary digital platform framework (700) of DAFT, is disclosed. This is a proprietary workstation solution, which has the specifications integrated into closed source code that works on a license model. Referring to FIG. 8, an exemplary DAFT's digital wireframe (800) and its capability to add unlimited modules, is disclosed.

The advantages or benefits of adopting DAFT are: (1) With one adoption in the workflow can support your discovery, preclinical and manufacturing stage bioassay or testing requirement, (2) Assays are embedded as modules pick and choose, (3) Module wise input and output data is stored in access restricted cloud computing block chain framework, (4) Assays are robust in predicting the concerns and requires no extrapolation to human species, (5) Assays can be performed in house Master banks of relevant hMPS will be supplied for exclusive usage, and (6) The prediction model will be trained with Reference drug related phenomics data as control panel.

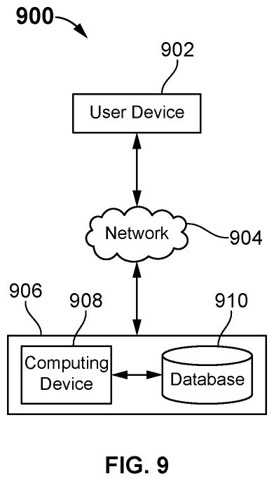

Referring to FIG. 9, the network environment 900 comprises one or more user devices 902. Each user device 902 is associated with a user. In one embodiment, the user device 902 is installed with a digital platform. In one embodiment, the digital platform may be an application software or mobile application or web-based application or software application. The system further comprises a network 904 and a neurovirulence evaluating system 906. In one embodiment, the user device 902 is enabled to access the neurovirulence evaluating system 906 via the network 904. In one embodiment, the user device 902 enables the user to access one or more services provided by the system. In one embodiment, the user device 902 is at least any one of a smartphone, a mobile phone, a tablet, a laptop, a desktop, and/or other suitable hand-held electronic communication devices. In one embodiment, the user device 902 comprises a storage medium in communication with the network 904 to access the neurovirulence evaluating system 906. In an embodiment, the network 904 could be Wi-Fi, WiMAX, wireless local area network (WLAN), satellite networks, cellular networks, private networks, and the like.

In one embodiment, the neurovirulence evaluating system 906 comprises a computing device 908 and one or more databases 910 in communication with the computing device 908. In one embodiment, the computing device 908 is a server. In one embodiment, the computing device 908 could be a cloud server. In one embodiment, the server could be operated as a single computer. In some embodiments, the computer could be a touchscreen and/or non-touchscreen and adopted to run on any type of OS, such as iOS™, Windows™, Android™, Unix™, Linux™, and/or others. In one embodiment, the plurality of computers is in communication with each other, via networks. Such communication is established via any one of an application software, a mobile application, a browser, an OS, and/or any combination thereof.

In one embodiment, the database 910 is in communication with the computing device 908 via the network 904. In one embodiment, the database 910 is accessible by the computing device 908. In another embodiment, the database 910 is integrated into the computing device 908 or separate from it. In some embodiments, the database 910 resides in a connected server or a cloud computing service. Regardless of location, the database 910 comprises a memory to store and organize certain data for use by the computing device 908.

In one embodiment, the computing device 908 comprises a processor and a computer-readable medium or memory unit coupled to the processor. The memory unit stores a set of instructions executable by the processor configured to test neurovirulence in the aliquots and to predict the risk involved. The memory unit could be RAM, ROM (including EPROM, EEPROM, PROM). In one embodiment, the user devices 902 are configured to access the services provided by the computing device 908 via the network 904. In one embodiment, the computing device 908 is configured to provide communication between the users in the digital platform.

In one embodiment, the computing device 908 is configured to, extract phenotype images acquired on hMPS platform or data source treated with vaccine aliquot of the batch; map the extracted data with the functional annotation (AI/ML/NLP (Neural)) with the reference data or training data sets; aggregate business rules for the extracted data, and visualize and analyze the extracted data by feeding into the software powered by machine learning algorithms that generate a score card and evaluates human neurovirulence test and cellular infiltration. In one embodiment, the phenotype data points are acquired from images supported by respective genotype profiles run by the reference data.

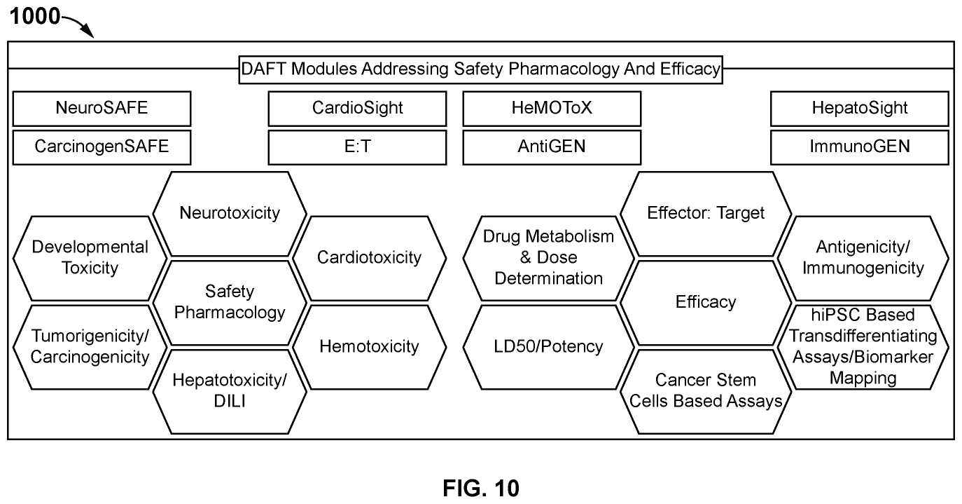

According to the present invention, referring to FIG. 10, DAFT combines in vitro or ex vivo platform composed of primary, progenitor cell/tissue configurations with AI/ML powered in silico protocols to assess either safety or efficacy assessments. DAFT is a New Approach Methodology (NAM) based technology that leverages relevant hMPS model (proprietary configurations) and AI/ML powered digital platform (proprietary software). DAFT's hMPS are human induced pluripotent stem cell (hiPSC) configured platform or hiPSC derived lineage specific cellular moieties or primary cells or primary progenitor cellular moieties mimicking human physiology or human adult stem cell-based configurations. The relevant hMPS model composition and the assaying protocol would dictate the use case of DAFT: For e.g., if hMPS is hiPSC derived neurons, it is NeuroSAFE as the modular assay for test predicting human neurovirulence or neurotoxicity. If hMPS is hiPSC derived cardiomyocytes, it is CardioSIGHT as the modular assay for test predicting cardiotoxicity. If hMPS is hiPSC derived hepatocytes milieu, it is HepatoSIGHT for test predicting hepatotoxicity and Drug Induced Liver Injury (DILI).

We claim:

1. A workstation solution for test predicting human safety concerns in a test agent, comprising:
a real-time platform or human MicroPhysiological Systems (hMPS) unit configured to incubate the test agent aliquots collected from a preparation, and
a digital platform with embedded artificial intelligence (AI) and machine learning (ML) tools, augmented with robotic process automation framework, wherein the digital platform with embedded artificial intelligence (AI) and machine learning (ML) tools, augmented with robotic process automation framework, is configured to predict safety (pharmacology) risks from one or more phenotypes, genotypes and proteotype data sets acquired from one or more test agents treated human MicroPhysiological Systems (hMPS) platform in a modular assay system,
wherein the digital platform is trained with one or more phenotypes, genotypes, proteotypes, biochemical data sets as one or more benchmark patterns and signals configured as one or more positive or negative controls to provide a bandwidth to the digital platform with embedded artificial intelligence (AI) and machine learning (ML) tools for detecting one or more anomalies in a real-time assaying, and wherein the workstation solution is a Digital Animal Free Testing (DAFT), which is a foundational scheme while the modular assay system is one of a derived application.

2. A workstation solution for test predicting efficacy measurements in a test agent, comprising:
a real-time platform or human MicroPhysiological Systems (hMPS) unit configured to incubate the test agent aliquots collected from a preparation, and
a digital platform with embedded artificial intelligence (AI) and machine learning (ML) tools, augmented with robotic process automation framework, wherein the digital platform with embedded artificial intelligence (AI) and machine learning (ML) tools, augmented with robotic process automation framework, is configured to predict safety (pharmacology) risks from one or more phenotypes, genotypes and proteotype data sets acquired from one or more test agents treated human MicroPhysiological Systems (hMPS) platform in a modular assay system,
wherein the digital platform is trained with one or more phenotypes, genotypes, proteotypes, biochemical data sets as one or more benchmark patterns and signals configured as one or more positive or negative controls to provide a bandwidth to the digital platform with embedded artificial intelligence (AI) and machine learning (ML) tools for measuring the analyzed insights in real-time assaying, and wherein the workstation solution is a Digital Animal Free Testing (DAFT), which is a foundational scheme while the modular assay system is one of a derived application.
